(19) United States
(12) Patent Application Publication
(10) Pub. No.: US 2008/0139520 A1
(43) Pub. Date: Jun. 12, 2008

(54) Altering cholesterol and fat uptake by novel allosteric inhibitors of pancreatic phospholipase A2

(76) Inventors: Mahendra K. Jain, Newark, DE (US); Rafael J. Apitz-Castro, Caracas (VE); Otto G. Berg, Daneryd (SE)

Correspondence Address: POTTER ANDERSON & CORROON LLP;ATTN: KATHLEEN W. GEIGER, ESQ., P.O. BOX 951, WILMINGTON, DE, 19899-0951, US

(21) Appl. No.: 11/894,871

(22) Filed: Aug. 21, 2007

Related U.S. Application Data
(60) Provisional application No. 60/839,346, filed on Aug. 22, 2006.

(51) Int. Cl. A61K 31/575; A61K 31/397; A61K 31/015; A61K 31/047; A61K 31/045; C07K 1/00; C12Q 1/42

(52) U.S. Cl. 514/177; 514/169; 514/182; 514/210.02; 530/345; 514/739; 514/738; 514/763; 435/18

(57) ABSTRACT

Disclosed herein are methods for regulation of fat and/or cholesterol uptake from the gastrointestinal tract and/or regulation of plasma fat and/or cholesterol levels comprising administering to a mammal in need thereof an effective amount of a regulator of pancreatic IB PLA2 functionality. Also disclosed herein are methods of regulating the function of a polypeptide of interest comprising inserting of the 62-66 loop region of a pancreatic IB PLA2 amino acid sequence into the polypeptide of interest; and administering an effective amount of a regulatory molecule that effects its regulation through said amino acid sequence. Further disclosed are novel bile salt compounds that regulate pancreatic IB PLA2. Methods for detecting altered pancreatic IB PLA2 function and methods for identifying an agent suitable for regulating pancreatic IB PLA2 enzyme functionality are also disclosed.

### CROSS-REFERENCE TO RELATED APPLICATION

This application claims priority from U.S. Provisional Application Ser. No. 60/839,346, filed Aug. 22, 2006, which is incorporated by reference herein in its entirety.

### FIELD OF THE INVENTION

The present disclosure relates to the use of bile salts and mimics thereof in the regulation of fat and/or cholesterol uptake through an interfacial pancreatic IB phospholipase A2 mechanism. The present disclosure also relates to novel bile salt compounds, mimics, analogs, and combinations thereof that similarly regulate fat and/or cholesterol uptake.

### BACKGROUND OF THE INVENTION

Gastrointestinal uptake of dietary fat and its metabolic consequences have taken the stage front and center as a human health concern [1-3]. In the digestive tract, fat emulsion encounters in stages the gastric, pancreatic and intestinal enzymes, including pancreatic IB phospholipase A₂ (PLA2) and other lipases whose kinetics of interfacial action are influenced by cosecreted bile salts and conjugates [4-6]. Not only the bile salt composition depends on the physiological and pathological state, but bile salts also influence other regulatory mechanisms for the lipid uptake and metabolism. Thus, bile salts or PLA2 are not obligatorily required for digestion of fat; however, they appear to play a role in the regulation of the fat uptake from high fat diet [1, 3, 7-10]. This is also consistent with the observation that competitive inhibitors of PLA2 lower the cholesterol and fat uptake [1, 3, 9].

Ezetimibe (Zetia®), a drug prescribed to lower absorption of dietary cholesterol, lowers the rate of hydrolysis of cholate containing dimyristoylphatidylcholine (DMPC) vesicles or of mixed-micelles of unsaturated phosphatidylcholine. Ezetimibe, however, is reported to produce potentially damaging side effects such as hepatotoxicity, cholestatic hepatitis, acute autoimmune hepatitis, myopathy, and modulation of monocytic raft assembly (see, e.g., [54-56]). Ezetimibe also acts on multiple gastrointestinal and systemic targets [56A].

There is thus a need for methods and compounds for regulating fat and/or cholesterol uptake from the intestine, but without the side effects and lack of specificity noted for ezetimibe. Applicants have addressed these needs as described and claimed below.

### SUMMARY OF THE INVENTION

One aspect relates to the design and selection of the allosteric regulators of pancreatic PLA2. Such compounds include but are not limited to bile salt and guggul compounds. Such compounds provide a basis for a method for up or down regulation of fat and/or cholesterol uptake from the intestine comprising administering to a mammal in need thereof an effective amount of a regulator of pancreatic IB PLA2 functionality and related sites for lipid homeostasis.

Another aspect relates to a method for regulation of plasma fat and/or cholesterol levels comprising administering to a mammal in need thereof an effective amount of a regulator of pancreatic IB PLA2 functionality.

A further aspect relates to a method of regulating the function of a polypeptide of interest comprising (a) inserting the 62-66 loop sequence of a pancreatic IB PLA2 sequence into a polypeptide of interest; and (b) administering an effective amount of a regulatory molecule that effects its regulation through said 62-66 loop region.

A further aspect is for a pharmaceutical composition for the regulation of uptake of fat and/or cholesterol from the gastrointestinal tract or for the regulation of plasma fat and/or cholesterol levels comprising an effective amount of a bile salt compound alone or in combination.

Another aspect relates to a method for determining the interfacial catalytic activity of pancreatic IB PLA2 enzyme in a sample comprising (a) combining said sample with a regulator of pancreatic IB PLA2 functionality; and (b) determining the amount of pancreatic IB PLA2 present as a function of the ability of the enzyme to digest lipids in the presence of said regulator.

An additional aspect is for a method for detecting altered pancreatic IB PLA2 function in a sample comprising (a) combining said sample with a regulator of pancreatic IB PLA2 functionality; and (b) determining the effectiveness of pancreatic IB PLA2 present in said sample as a function of the ability of the enzyme to digest lipids in the presence of said regulator.

A further aspect relates to a method for identifying an agent suitable for regulating pancreatic IB PLA2 enzyme functionality comprising (a) combining said agent with pancreatic IB PLA2; and (b) determining the ability of the agent to regulate pancreatic IB PLA2 functionality as a function of the ability of the enzyme to digest lipids in the presence of said agent.

Other aspects of the regulation of PLA2 functionality relate to IB PLA2 expressed in other tissues. For example, IB PLA2 is expressed in lung under certain conditions, presumably to modify the lung surfactant. If the expression is in response to or is a consequence of a signal for the pathology, regulation of IB PLA2 could control or treat the underlying condition.

A further aspect relates to a compound of the formula:

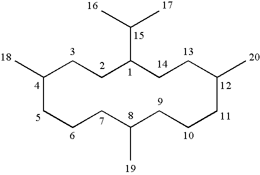

having modifications selected from the group consisting of double bonds at positions 3-4, 7-8, 11-12, and 15-16; double bonds at positions 2-3, 4-5, 7-8, and 11-12; double bonds at positions 34, 7-8, and 11-12; and double bonds at positions 3-4, 7-8, and 11-12; said cembrene further optionally comprising a hydroxyl group, carbonyl group, acetyl group, and/or calixerene ring structure with fused aromatic rings at any of the twenty carbon positions.

Other aspects and advantages will become apparent to those skilled in the art upon reference to the detailed description that hereinafter follows.

### BRIEF DESCRIPTION OF THE FIGURES

FIG. 1A. Structures of (right) ezetimibe and (left) bile salts and conjugates. Hydroxyl groups at 3, 7 and 12 are normally in a orientation. Bile acids can be conjugated with, for example, taurine (shown) or glycine (—NH—CH₂—COO⁻). B. Structure of masticadienoic acid (R⁵=(═O)) and masticadienolic acid (R⁵=α-OH, H). C. Structure of guggulsterone (Z and E enantiomers). D. The carbon skeleton of cembrenes. Naturally occurring cembrenes typically contain 3 to 5 double bonds with none to as many as half a dozen oxygen substituents. Cembrenes from guggul are: cembrene #6 has Δ(3, 7, 11, 15), cembrene #14 has Δ(2, 4, 7, 11), cembrene #15 has 1-OH with Δ(3, 7, 11), cembrene #16 has 2-OH with Δ(3, 7, 11). (Number in parenthesis indicates double bond position.)

FIG. 2. Effect of added 0.025 mM deoxycholates (DOC) (cheno=taurocheno-DOC; tauro=tauro-DOC; glyco=glyco-DOC) on the reaction progress curve for the hydrolysis of 1 mM sonicated DMPC vesicles with 0.2 μg WT (wild type) pig PLA2 at 24° C. The unmarked reaction progress with a delay of about 50 minutes is obtained in the absence of bile salt. For the rates above, 0.025 mM bile salts (FIG. 3) the delay was <1 min. These initial rates are reliable quantitative measures of the regulation of the interfacial catalytic turnover by bile salt compounds.

FIG. 3. Effects of bile salt concentration on the PLA2 catalyzed apparent initial rate of hydrolysis of sonicated DMPC vesicles in the presence of (from top at about 0.07 mM) cholate, deoxycholate, glycocholate, glyco-DOC, tauro-DOC, taurocheno-DOC, urso-DOC and taurourso-DOC. Note that a major difference between these bile salts is in their effect on the falling phase. Also, the cholate activated rate is lowered by the bile salts that show pronounced falling phase, and thus regulate the PLA2 activity (results not shown).

FIG. 4. The initial rate of hydrolysis of 1 mM sonicated DMPC vesicles with (unfilled symbols and dashed line) cholate or (filled symbols and continuous line) TCDOC concentration at (squares) 16° C., (triangles) 24° C. and (circles) 29° C. Crosses show the effect of TCDOC on the hydrolysis of 1,2-dimyristoyl-sn-glycero-3-phosphomethanol (DMPM) vesicles at 24° C. Note that both of the axes are logarithmic.

FIG. 5A. Apparent initial rate of hydrolysis of sonicated DMPC (1 mM) vesicles by (62-66)-loop deleted pig pancreatic IB ΔPLA2 with added (squares) cholate or (triangles) TCDOC. B. The change in the observed initial rate of hydrolysis of 1 mM DMPC sonicated vesicles by human IB PLA2 with added (squares) cholate or (triangles) TCDOC. The rate of hydrolysis of DMPC+0.05 mole fraction cholate changes with added (circle) TCDOC or (diamonds) ezetimibe.

FIG. 6. Concentration (log scale) dependence (squares) masticadienolic acid, (diamonds) ezetimibe, and (circles) TCDOC on the normalized rate of hydrolysis by pig pancreatic IB PLA2 of 1 mM DMPC vesicles containing 0.05 mM cholate.

FIG. 7. The change in the Trp emission intensity at 333 nm (excitation 280 nm) of a mixture of 1 μM PLA2, 1 mM 1,2-ditetradecyl-sn-glycero-3-phosphocholine (DTPC) vesicles and 0.05 mM products of hydrolysis of DMPC (1:1 myristic acid+1-myristoylphosphatidylcholine) on the addition of (squares) cholate or (circles) TCDOC.

FIG. 8. PCU concentration dependent change in (relative) inactivation time of PLA2 by p-nitrophenacylbromide (unfilled squares) in buffer containing 1.3 mM EGTA or (filled squares) the 0.5 mM calcium containing buffer alone or with 0.2 mM (unfilled squares) TCDOC or (crosses) 0.2 mM cholate.

FIG. 9A. Exothermic heat change on the titration of 8.5 μM PLA2 with (circles) cholate or (squares) TCDOC. No heat change is seen with cholate. Only the TCDOC curve is shown. Fit parameters obtained by taking the depletion of the bile salt titrant into consideration are: TCDOC (K_{EB} 9 μM, ΔH-12 kcal/mole, S, 3.5 cal/mole/deg). From the fits (results not shown) for tauroursodeoxycholate (TUDOC; K_{EB}<2 μM, ΔH-16 kcal/mole); ursodeoxycholate (UDOC; K_{EB} 37 μM, ΔH-12 kcal/mole); and taurodeoxycholate (TDOC; K_{EB} 38 μM, ΔH-14 cal/mole). B. TCDOC concentration dependent change in the Trp emission intensity from the PLA2 mutants at 355 nm from (up triangles, top) W3F/R6W, (squares) WT/3W, (diamonds) W3F/M20W, and (inverted triangle, bottom) W3F/K10M. The fits are for a single hyperbola. The fit parameters for these and other mutants are summarized in Table 1.

FIG. 10A. Effect of bile salts on the RET signal at 445 nm (excitation 280 nm) from an equimolar mixture of PLA2 and trimethylamino-diphenylhexatriene (TMA-DPH) (1 μM each) in 2 mM calcium and 10 mM Tris at pH 8.0. Rank order for the apparent bile salt concentration for 50% displacement I_{B}(50) is (from top): cholate>DOC>glycoDOC>urso-DOC>tauro-DOC>taurocheno-DOC>taurourso-DOC. B. Decylsulfate concentration dependent change of the RET emission intensity from 1 μM TMA-DPH mixed with 1 μM PLA2 at pH 6.9 in EGTA buffer (diamonds) alone or containing 0.2 mM (squares) deoxycholate.

FIG. 11A. Enthalpy (arbitrary on the same scale, uncorrected for the dilution of decylsulfate) change during isothermal calorimetric titration with decylsulfate of 8.5 μM PLA2 (circles) alone, or in the presence of 0.2 mM (triangle) cholate or (squares) TCDOC. B. Decylsulfate concentration dependence of the Trp-emission signal at 333 nm from PLA2 (circles) alone, or in the presence of 0.2 mM (diamonds) cholate or (triangles) TCDOC. Fit no additive K1 0.06/0.24/1.8, and +cholate 0.05/0.10/1.6.

FIG. 12. Fitted curves for a model with ideal partitioning of bile salt in the substrate layer, and E*B in the interface competes for lowering the interfacial turnover rate mediated by E*S.

FIG. 13. Fitted-curves for a model with non-ideal partitioning of bile salt in the substrate layer, and EB in the aqueous phase competes for lowering the interfacial turnover rate mediated by E*S.

FIG. 14. The mole fraction dependence of the rate lowering effect of cembrene #15 (from guggul resin) on the human pancreatic PLA2 catalyzed hydrolysis of DMPC+cholate. See also Table 1 for related results.

### DETAILED DESCRIPTION OF THE INVENTION

Applicants specifically incorporate the entire content of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Here, Applicants show that the allosteric inhibitors and activators of the interfacial turnover by pancreatic IB phospholipase A2 (PLA2) offer a novel mechanism for the regulation of cholesterol and fat uptake. Uptake of dietary fat and cholesterol is controlled by the initial interfacial action of pancreatic IB phospholipase A2 (PLA2) on phospholipid monolayer of dietary fat emulsion containing bile salts. Conventional view is that the gastrointestinal role of bile salts is limited to their detergent action to co-micellize dietary fat. Based on critical evidence that addresses this issue, such effect is unlikely to be significant. In this application, Applicants further demonstrate that, depending on their structure, bile salts not only allosterically activate PLA2 but certain bile salts also lower the rate activated by other bile salts. This key result physiologically complements with the emerging consensus about the regulatory action of bile salts on farnesoid X receptor (FxR) that controls the transcription of wide ranging proteins involved in the long term homeostasis [56E]. In other words, specific effects of bile salts on PLA2 coupled with their agonist or antagonist effects on FxR give rise to several possible treatments for the regulation of short or long terms lipid and cholesterol homeostasis. Such possibilities include treatment with modulators with specific activating or inhibiting effect only on PLA2, or only on FxR. In addition, treatment with modulators that have similar or opposing effect on PLA2 and FxR would have specific desirable effects or alleviate undesirable effect of high fat diet. These results also provide insights about the conditions and models (both in vivo and in vitro) to characterize allosteric regulation by bile salt compounds. The assay conditions may favor the consequences at the PLA2 or the FxR end of the metabolic pathway. Thus, the PLA2 rate lowering bile salt compounds are a new class of drugs to regulate fat and cholesterol homeostasis based on a novel mechanism. The main target of these compounds is the gastrointestinal tract, and they are likely to have favorable regulatory and pharmacokinetic profiles without direct systemic effect unless desired so.

Pancreatic PLA2 is cosecreted with bile salts. Applicants show herein that bile salts have complex kinetic effects on the PLA2 catalyzed interfacial hydrolysis of zwitterionic phospholipids. For example, bile salts influence the PLA2 catalyzed reaction progress for the hydrolysis of zwitterionic dimyristoylphatidylcholine (DMPC) vesicles. Here, a surprising result is that most bile salts show a rate increase that depends on the source of the enzyme and the structure of the bile salt. Also, some bile salts show a biphasic effect, that is the rate increase is followed by a rate decrease at a higher bile salt concentration. The rate increase is due to enhanced binding of PLA2 to the anionic interface, and due to interfacial k_{cat}*-activation by charge compensation of certain cationic residues on the bound enzyme. The rate decrease is attributed to the allosteric inhibition of the interfacial catalytic turnover.

One of the unique structural characteristics of group IB PLA2s is the pentapeptide pancreatic loop at amino acid residues 62-66 of the protein (consensus sequence K(F/V/)L(V/I/L)D). The 62-66 loop has been shown to be involved in interfacial binding [50], and it has been theorized that the 62-66 loop may also be involved in substrate recognition [51] and therefore in the interfacial allosteric regulation of PLA2.

Applicants also demonstrate a specific effect of the structure of bile salt compounds. For example, cholate shows only a rate increase, whereas taurochenodeoxycholate (TCDOC) shows a rate increase and then a rate decrease at higher concentration. With 1 mM DMPC vesicles, the rate increase and decrease is complete at <0.12 mM TCDOC where the bilayer organization remains intact. The rate lowering effect of TCDOC observed with pancreatic PLA2 is not observed with the (62-66)-loop deleted ΔPLA2 mutant, or with the Naja venom enzyme in which the 62-66 loop is evolutionarily deleted. TCDOC and ezetimibe are neither competitive inhibitors nor bind to the active site of PLA2. Relative efficacies of ezetimibe, TCDOC and other rate lowering compounds are significantly different, and thus provide a basis for selective targeting.

As developed below, the rate lowering effect of bile salts and guggul compounds is highly specific, which also correlates well with their hypolipidemic action. The regulatory consequences of the monophasic and biphasic kinetic effects of bile salts are far reaching because modest amounts of bile salt compounds could change the gastrointestinal PLA2 activity by more than 1000-fold. Such kinetic regulation by bile salt compounds of the hydrolysis of phosphatidylcholine on the surface of dietary fat emulsion particles could in turn regulate fat metabolism and cholesterol homeostasis. Thus, suitable bile salt compounds are of general interest because the guggul compounds are antagonist ligands for FxR for which bile salts are natural regulators for the transcription of the regulators for cholesterol homeostasis in liver, kidney and small intestine [56B]. This effect may also enhance the hypolipidemic action of guggul and certain bile salts because FxR is an important regulator of lipid homeostasis.

### I. DEFINITIONS

In the context of this disclosure, a number of terms shall be utilized.

The terms “effective amount”, “therapeutically effective amount”, and “effective dosage” as used herein, refer to the amount of a PLA2 regulator compound that, when administered to a mammal in need, is effective to at least partially ameliorate a condition from which the mammal is suspected to suffer.

The term “mammal” refers to a human, a non-human primate, canine, feline, bovine, ovine, porcine, murine, or other veterinary or laboratory mammal. Those skilled in the art recognize that a therapy which reduces the severity of a pathology in one species of mammal is predictive of the effect of the therapy on another species of mammal. The skilled person also appreciates that credible animal models of fat and/or cholesterol uptake are known (see, e.g., [1, 3, 9, 53] (and references therein)).

“Pancreatic IB phospholipase A2” or “pancreatic IB PLA2” refers to the pancreatic IB phospholipase A2 protein as defined by its conserved amino acid coding sequence in an active or native structural conformation. Nucleic acid sequences encoding pancreatic IB PLA2 have been cloned and sequenced from numerous organisms. Representative organisms and GenBank® accession numbers for pancreatic IB PLA2 sequences therefrom include the following: human (Homo sapiens, NP_{—}000919), mouse (Mus musculus, NP_{—}035237), cow (Bos taurus, NP_{—}777071), pig (Sus scrofa, NP_{—}001004037), rat (Rattus norvegicus, NP_{—}113773), sheep (Ovis aries, P14419), rabbit (Oryctolagus cuniculus, Q7M334), and guinea pig (Cavia porcellus, P43434).

### II. REGULATION OF FAT AND/OR CHOLESTEROL UPTAKE

Applicants disclose herein that gastrointestinal PLA2 and bile salts regulate fat and cholesterol uptake by controlling the phospholipid monolayer on fat emulsion particles in high fat diet. The fat derived calories come from triglycerides, and, as minor dietary component, phospholipids may account for <5%. Dietary fat emulsion droplets are surrounded by phospholipid monolayer. PLA2-catalyzed interfacial hydrolysis of phospholipid would decrease the surface area and therefore change particle size and dispersity. Decrease in the surface area would increase the emulsion particle size to retain nonpolar triglyceride and cholesterol esters in the core of the particle. Such changes in the surface to volume ratio would also change susceptibility and accessibility of the emulsion components to the hydrolytic enzymes and the receptors for their uptake. All such interfacial effects control the binding and activation of lipolytic and transacylating enzymes to regulate accessibility, metabolism and absorption of the emulsion components. In effect, the PLA2 and bile salt mediated changes could regulate the emulsion behavior as well as uptake and secretion of fat emulsion particles at appropriate stages in the gastrointestinal tract. This mechanism also predicts that cholesterol and fat uptake would depend on the ratio of dietary triglyceride/phospholipid.

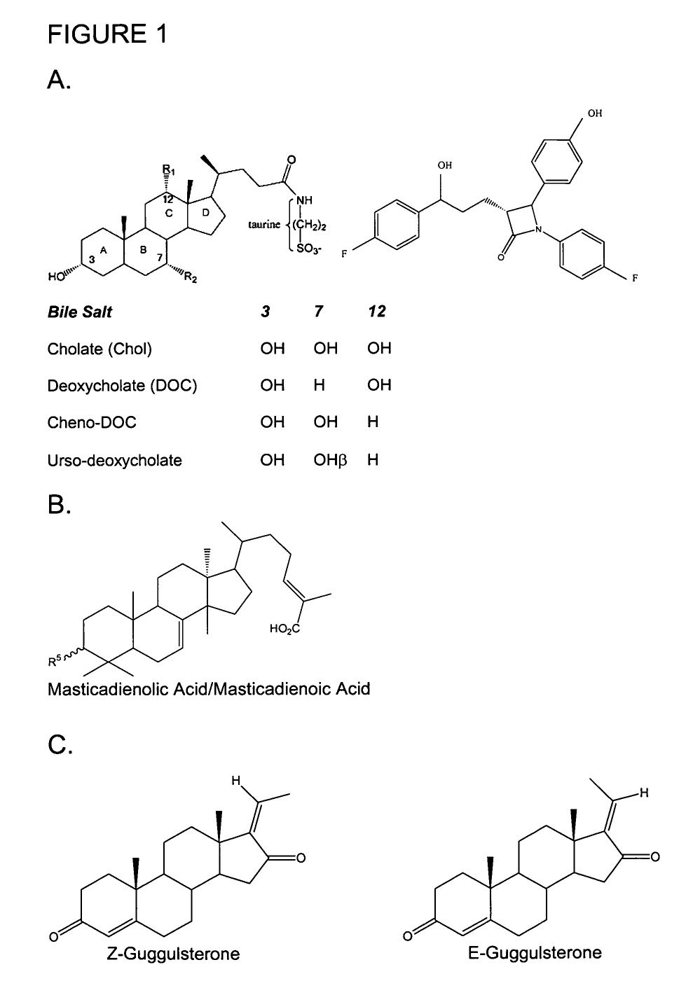

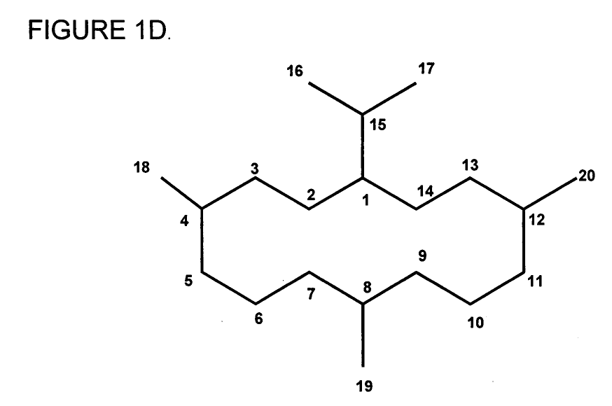

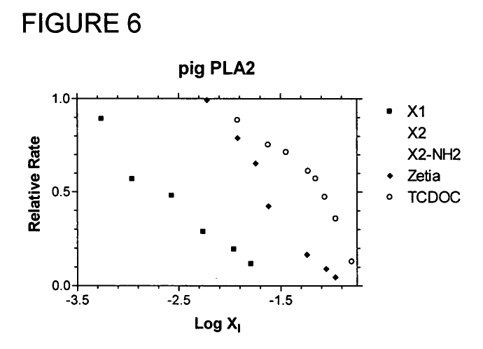

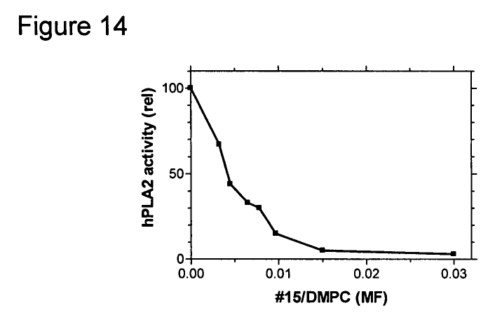

Anionic amphiphiles including bile salts increase the rate of interfacial hydrolysis of phosphatidylcholine vesicles by PLA2 [11-15] and the fractions of the enzyme bound to the interface [16-18]. Here, Applicants show that certain bile salt compounds also lower the rate of hydrolysis of phosphatidylcholine. These results have far reaching implications because the mixture of gastrointestinal bile salts and conjugates is regulated and is species specific, so also the effects of the bile compounds. Thus, depending on the concentration and structure of bile salt (FIG. 1), the pancreatic PLA2 catalyzed rate increase and decrease would have a wide range. One aspect is that the rate decrease at higher interfacial mole fraction of certain bile salts such as TCDOC is due to formation of a specific EB complex with PLA2 in the aqueous phase, which may or may not bind to the substrate interface. Effects of TCDOC are not seen with the (62-66)-loop deleted mutant, suggesting that the 62-66 loop of pancreatic PLA2 has evolved for the interfacial kinetic regulation by bile salts. Ezetimibe (FIG. 1) also lowers the PLA2 catalyzed rate of interfacial hydrolysis of DMPC in the presence of cholate (FIG. 6). Ezetimibe is prescribed to lower intestinal cholesterol absorption [8, 19], although its mechanism of action is not established [8, 20-22]. Together, kinetic regulation of the PLA2 catalyzed hydrolysis of dietary phospholipid by bile salts could control gastrointestinal uptake of excessive dietary fat and cholesterol. As shown in Table 1 and FIG. 14 this conclusion is also supported by the PLA2 regulatory effect of novel compounds, from guggul.

The mono- and biphasic effects of bile salts are physiologically relevant. Although the rate increase by anionic bile salts is expected [4], the rate lowering, effect of a subgroup of certain bile salts, such as TCDOC, is surprising. Other bile salts demonstrating the biphasic effect include, for example, TDOC (taurodeoxycholate), glycocholate, GDOC (glycodeoxycholate), and TUDOC (tauroursodeoxycholate).

Applicants have used TCDOC for the detailed characterization of the biphasic effect and its mechanics basis. Significance of the rate lowering effect of TCDOC with pancreatic PLA2 is further supported by the fact that the cholesterol-lowering drug ezetimibe or certain plant components also lower the PLA2 catalyzed rate hydrolysis of DMPC in the presence of cholate.

Applicants previously have shown that the rate increase with cholate is not due to the micellization of DMPC [13] but due to the enhanced binding of pancreatic PLA2 to zwitterionic interface containing anionic amphiphiles [12, 13, 39] and also the allosteric k_{cat}*-activation of the bound enzyme on the anionic interface by the charge compensation of certain cationic surface residues [38]. The unexpected finding disclosed herein is that the rate-lowering effect depends on the 62-66 loop that is found only in pancreatic PLA2 [4]. This loop is part of the regulatory R-site involved in the coupling of the i-face and active site events presumably responsible for the interfacial activation of PLA2.

Specificity and stability of the EB complex of bile salts with pancreatic PLA2 provide kinetic and structural insights into the regulatory role for diverse bile salts by increasing and lowering the pancreatic PLA2 catalyzed rate. Not only is the regulatory significance of the differences in the rate lowering effect of bile salts far reaching, but results with ezetimibe and other components also suggest that their effect on the absorption of dietary cholesterol may also be based on a comparable effect of bile salts. Pancreatic PLA2 and cosecreted bile salts and conjugates may have coevolved to regulate the hydrolysis of zwitterionic interface of the fat emulsion in the gastrointestinal environment. Dietary fat (triglyceride and cholesterol) emulsion particles are stabilized by a surface monolayer of phospholipids. Thus, a change in the amount of phospholipid, for example by PLA2-catalyzed hydrolysis, would lower the monolayer area compensated by an increase in the particle size. This would certainly influence the kinetics of action of gastrointestinal lipases and acyltransferases. Such a change in the processing of the dietary lipids sequestered in the emulsion particles would therefore influence gastrointestinal absorption of triglycerides and cholesterol(esters). This suggestion is consistent with the observation that ezetimibe [8, 19] as well as the competitive inhibitors of pancreatic PLA2 [1, 9] lower the gastrointestinal uptake of fat and cholesterol with other physiological consequences [3, 10, 20].

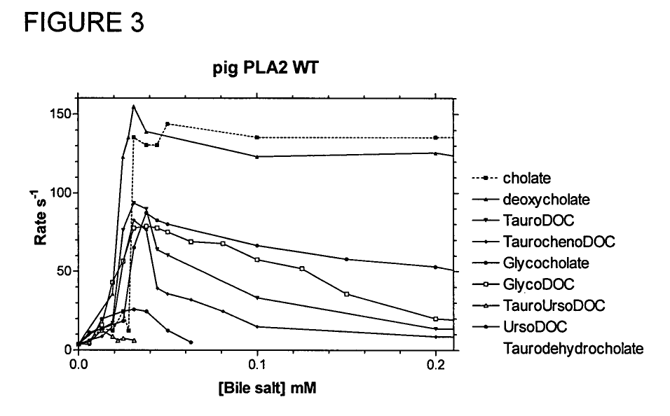

Applicants herein disclose that PLA2 and bile salts regulate the fat uptake from the high fat diet. As is apparent in pancreatitis, cholestasis, formation of gallstone, and the outcomes of gall bladder surgery, secretion of PLA2 or bile salts are not obligatorily required for fat uptake and digestion. The result that ezetimibe and TCDOC have similar kinetic effects on the rate of PLA2 catalyzed hydrolysis of DMPC vesicles raises the possibility that, while PLA2 and cholate may promote fat uptake, the bile salts that lower the PLA2 catalyzed rate may be the natural regulators of the fat uptake (FIG. 3). For example such useful compounds include bile salt derivatives of the formula:

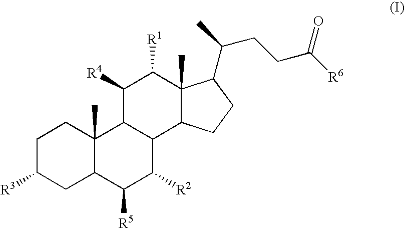

wherein R¹ is H, OH, O, or Ac; R² is H, OH, O, or Ac; R³ is OH or O; R⁴ is H, OH, or O; R⁵ is H, OH, O, or Ac; and R⁶ is COOH, CONH₂, SO₄, PO₄, CO-taurine, CO-glycine, CO—NH—(CH₂)ₙ-anion glucuronate, wherein the anion is COOH, —O-phosphate, C-phosphate, O-sulfate, or —C-sulfate and n is an integer in the range of 8 to 12. Standard methods (for example, see [56D]) can be used to produce these Formula (I) derivations of natural bile salts.

Other classes of bile salt compounds with similar activity but different selectivity profiles are also obtained from sterols and terpenes including masticadienoic acid, masticadienolic acid, cembrenes, guggulosterone and their structural analogs. The common three-dimensional structural features of these lead compounds can be used to design novel bile salt compounds with cembrene, calixerene, steroid or terpene skeletons. Cembrene compounds, as seen in FIG. 1D are preferred, with said compounds optionally having double bonds and/or oxygen substitutes anywhere on the structure. Particularly preferred cembrene compounds have modifications selected from the group consisting of double bonds at positions 3-4, 7-8, 11-12, and 15-16; double bonds at positions 2-3, 4-5, 7-8, and 11-12; double bonds at positions 3-4, 7-8, and 11-12; and double bonds at positions 3-4, 7-8, and 11-12; said cembrene further optionally comprising a hydroxyl group, carbonyl group, acetyl group, and/or calixerene ring structure with fused aromatic rings at any of the twenty carbon positions.

### III. MODULATION OF POLYPEPTIDE FUNCTION VIA THE 62-66 LOOP REGION

Another aspect is for a method of regulating the interfacial function of a phospholipase A2 polypeptide of interest wherein the polypeptide comprises a 62-66 loop region of a pancreatic IB PLA2 amino acid sequence that has been inserted into the polypeptide of interest through any biological or chemical synthesis method as are well known to those of ordinary skill in the art (see, for example, [52]). For example, polypeptides containing a 62-66 loop region can be purified from cells that have been altered to express it (i.e., recombinant) or synthesized using polypeptide synthesis techniques that are well known in the art. In one embodiment, the polypeptide is produced by recombinant DNA methods. In such methods, a nucleic acid molecule encoding the polypeptide is cloned into an expression vector and expressed in an appropriate host cell according to known methods in the art. The polypeptide is then isolated from cells using polypeptide purification techniques well known to those of ordinary skill in the art. Alternatively, the polypeptide or fragment can be synthesized using peptide synthesis methods well known to those of ordinary skill in the art.

Preferred polypeptides that are modified with a 62-66 loop region from pancreatic IB PLA2 are phospholipases, preferably phospholipase A2s, which do not contain a 62-66 loop region. In this way, a phospholipase having, for example, an insertion mutation of a 62-66 loop region is substantially identical to the wild-type phospholipase polypeptide from which the mutant has been constructed, with the addition of a pancreatic IB PLA2 62-66 loop region located in the mutant. Mutants constructed in such fashion should retain wild-type phospholipase activity while gaining biphasic responsiveness to bile salts as described elsewhere herein. Methods for mutagenesis and nucleotide sequence alterations are well known in the art (see, e.g., U.S. Pat. No. 4,873,192; [57-59]; and the references cited therein).

### IV. ADMINISTRATION OF REGULATORS OF PANCREATIC IB PLA2

Known bile salt compounds, novel bile salt compounds, and mimics thereof (collectively “bile salt compounds”) can be administered to subjects in a biologically compatible form suitable for pharmaceutical administration in vivo. By “biologically compatible form suitable for administration in vivo” is meant a form of the bile salt compound to be administered in which any toxic effects are outweighed by the therapeutic effects of the compound. The term subject is intended to include living organisms in which an immune response can be elicited, for example, mammals. Administration of a bile salt compound as described herein can be in any pharmacological form including a therapeutically active amount of a bile salt compound alone, in combination with a pharmaceutically acceptable carrier, or in combination with competitive inhibitors of PLA2 that are known to be non-toxic.

In a preferred embodiment, there are three kinds of potential applications (uses) of the bile salt compounds: (1) to lower the uptake of fat and cholesterol (of which a useful combination would be, for increasing the gastric emulsion size, the rate-lowering compounds); (2) for (decreasing the gastric emulsion size) increasing the fat uptake the combination would be phospholipids and rate increasing bile salts and, optionally, engineered pancreatic PLA2 for the species; and (3) to increase the uptake of fat and cholesterol by increasing the proportion of bile salts that increase the rate. A possible concern with administration of bile salt compounds is emulsion stability in acid medium. To alleviate this concern, zwitterionic or non-ionic emulsions/emulsifiers may be desirable (or, in another embodiment, using a coating that does not disintegrate in the acidic stomach).

Both ezetimibe and guggul components lower the PLA2 catalyzed rate. However, their profiles for lowering the fat and cholesterol uptake appear to be different. For example, ezetimibe is reported to lower the gastrointestinal absorption of dietary cholesterol but not the uptake of other lipidic components. As noted above, ezetimibe is reported to produce potentially damaging side effects such as hepatotoxicity, cholestatic hepatitis, acute autoimmune hepatitis, myopathy, and modulation of monocytic raft assembly (see, e.g., [54-56]). Ezetimibe also acts on multiple gastrointestinal and systemic targets [56A]. At least some of the effects of ezetimibe are attributed to its glucuronylated form, which may also mediate other systemic effects including potentiation of the effect of statins by inhibiting its efflux [29, 69-71]. Also at concentrations >20 μM, well above its aqueous solubility limit, ezetimibe is reported to bind to acylCoA-cholesterol acyltransferase, apical drug efflux pumps, aminopeptidase N, and to a Nieman-Pick C1 like protein which appears to be involved in the intestinal cholesterol uptake.

On the other hand, therapies with bile salt compound, including those from the PLA2 rate lowering guggul components, described herein are likely to have very desirable hepatic recirculation and secretion with minimal systemic exposure. As such, the potential for side effects in bile salt compound treatment should be minimal. Additionally, similar to cholesterol-lowering plant steroids such as, for example, [67-68], bile salt compounds disclosed herein are not likely to become part of atherosclerotic plaques.

A therapeutically effective amount of a bile salt compound may vary according to factors such as the disease state, age, sex, and weight of the individual, and the ability of the compound to elicit a desired response in the individual. Dosage regime may be adjusted to provide the optimum therapeutic response. For example, several divided doses may be administered daily, or the dose and composition may be proportionally reduced as indicated by the exigencies of the therapeutic situation, or only a ‘morning after pill’ following a binge with fatty foods. Bile salt compounds are also compatible with common foods.

The therapeutic or pharmaceutical compositions can be administered by any suitable route known in the art including, for example, intravenous, subcutaneous, intramuscular, transdermal, intrathecal, oral, rectal, or intracerebral or administration to cells in ex vivo treatment protocols. Administration can be either rapid as by injection or over a period of time as by slow infusion or administration of slow release formulation. For regulation of fat and/or cholesterol uptake, administration of the therapeutic or pharmaceutical compositions of the present invention can be performed. For example, since the pharmacological target of these compounds is in the gastrointestinal tract, the most desirable method of administration would be oral. Overall, for most applications the most convenient bile salt compounds can be orally administered as pills alone or in combination with dietary food emulsifiers such as phospholipids or fats.

Bile salt compounds can be stably conjugated with sugars or linked to a polymer such as polyethylene glycol to obtain desirable properties of solubility, stability, half-life, and other pharmaceutically advantageous properties (see, e.g., [47, 48]).

A bile salt compound may be incorporated and conjugated with a carrier moiety such as a liposome that is capable of delivering the compound into the cytosol of a cell. Such methods are well known in the art (see, e.g., [49]). Alternatively, the compound can be delivered directly into a cell by microinjection.

The bile salt compounds are usefully employed in the form of pharmaceutical preparations. Such preparations made with media and agents for pharmaceutically active substances are well known in the art. One preferred preparation utilizes a vehicle of physiological saline solution without or with glucose and other nutrients. It is contemplated that other pharmaceutically acceptable carriers such as physiological concentrations of other non-toxic amphiphiles and salts may also be used. As used herein “pharmaceutically acceptable carrier” includes any and all solvents, dispersion media, coatings, antibacterial and antifungal agents, isotonic and absorption delaying agents, and the like. Except insofar as any conventional media or agent is incompatible with the active compound, use thereof in the therapeutic compositions is contemplated. Supplementary active compounds can also be incorporated into the compositions. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous.

The carrier can also contain other pharmaceutically-acceptable coatings and excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion by continuous or periodic infusion.

Bile salt compounds may be used individually or in combination and with other bile salt compounds or other treatments, such as ezetimibe, cholate, a statin (e.g., cerivastatin, fluvastatin, atorvastatin, lovastatin, pravastatin, simvastatin), nicotinic acid, a fibrate (e.g., bezafibrate, ciprofibrate, clofibrate, gemfirozil, fenofibrate), a bile acid-binding resin (provided, however, that the bile acid-binding resin is capable of discriminating between cholate and bile acid compound of the present disclosure), or a pancreatic IB PLA2 competitive inhibitor (see, e.g., [24, 36, 60-64]), as may be conventionally employed and as may be moderated for use in conjunction with the bile salt compounds.

Dose administration can be repeated depending upon the pharmacokinetic parameters of the dosage formulation and the route of administration used.

It is also provided that certain formulations containing the bile salt compounds are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents, or flavoring agents. The compositions may be formulated so as to provide rapid, sustained, or delayed release of the active ingredients after administration to the patient by employing procedures well known in the art. The formulations can also contain substances that diminish proteolytic degradation and/or substances which promote absorption such as, for example, surface active agents.

It is especially advantageous to formulate compositions in dosage unit form for ease of administration and uniformity of dosage. “Dosage unit form” as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms disclosed herein are dictated by and directly dependent on (a) the unique characteristics of the active compound and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The calculated dose will also be dependent upon the particular route of administration selected. Further refinement of the calculations necessary to determine the appropriate dosage for treatment is routinely made by those of ordinary skill in the art. Such calculations can be made without undue experimentation by one skilled in the art in light of the pancreatic IB PLA2 activity disclosed herein in assay preparations of target cells. Exact dosages are determined in conjunction with standard dose-response studies. One of the key variables here would be the dietary triglyceride/phospholipid ratio. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Toxicity and therapeutic efficacy of such compounds can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and thereby reduce side effects. Available information suggests that LD₅₀/ED₅₀ ratio for oral dosage of bile salts is very high (possibly >100).

In another embodiment, the ability of a bile salt compound to modulate pancreatic IB PLA2 activity in a subject that would benefit from modulation of the activity of the pancreatic IB PLA2 can be measured by detecting an improvement in the condition of the patient after the administration of the compound. Such improvement can be readily measured by one of ordinary skill in the art using indicators appropriate for the specific condition of the patient. Monitoring the response of the patient by measuring changes in the condition of the patient is preferred in situations were the collection of biopsy materials would pose an increased risk and/or detriment to the patient.

Furthermore, in the treatment of disease conditions, compositions containing bile salt compounds can be administered exogenously, and it would likely be desirable to achieve certain target levels of bile salt compounds in any desired tissue compartment, or in the affected tissue. It would therefore be advantageous to be able to monitor the levels of bile salt compounds in a patient or in a biological sample, including a tissue biopsy sample obtained from a patient. Accordingly, the present invention also provides methods for detecting the presence of bile salt compounds in a sample from a patient.

### V. ASSAYS

Another aspect of Applicants' disclosure is for assays utilizing pancreatic IB PLA2 in combination with regulators thereof. In one embodiment, the concentration of pancreatic IB PLA2 enzyme in a sample can be determined by combining the sample with a regulator of pancreatic IB PLA2 functionality and determining the amount of pancreatic IB PLA2 in the sample by measuring PLA2 digestion of lipids.

Measurement of PLA2 activity, and in turn PLA2 concentration in a sample, is a function of the regulator's effect on PLA2 activity. For example, as disclosed herein, cholate has a mono-phasic and TCDOC has a concentration-dependent, biphasic effect on the rate of PLA2 hydrolysis of lipids. By measuring the rate of lipid hydrolysis in the presence of a known concentration of bile salt, for example cholate, the functional specific activity of PLA2 in the sample is readily calculated. The effect of the rate lowering bile salt compounds can be assayed in this mixture. Note that cholate does not compete with the PLA2 rate-lowering regulatory bile salt compounds.

Another embodiment is for detecting altered pancreatic IB PLA2 function in a sample. Similar to the PLA2 concentration assay described above, measurement of PLA2 activity is a function of the regulator's effect on PLA2 activity. However, in this assay, a specific activity of PLA2 is present in the sample, and altered PLA2 activity is a function of comparing the rate of lipid hydrolysis by PLA2 in the sample to a baseline rate of PLA2 activity.

In many drug screening programs that test libraries of modulating agents and natural extracts, high throughput assays are desirable in order to maximize the number of modulating agents surveyed in a given period of time. Assays that are performed in cell-free systems, such as may be derived with purified or semi-purified proteins, are often preferred as “primary” screens in that they can be generated to permit rapid development and relatively easy detection of an alteration in a molecular target that is mediated by a test modulating agent. Moreover, the effects of cellular toxicity and/or bioavailability of the test modulating agent can be generally ignored in the in vitro system, the assay instead being focused primarily on the effect of the drug on the molecular target as may be manifest in an alteration of binding affinity with upstream or downstream elements.

Assays can be used to screen for modulating agents including those which are either agonists or antagonists of the normal gastrointestinal function of pancreatic IB PLA2. For example, provided herein is a method in which the effect of the test compound on pancreatic IB PLA2 activity, as measured by a method described herein, can be quantitatively determined to thereby identify a compound that modulates the activity of pancreatic IB PLA2. A statistically significant change, such as a decrease or increase, in the level of pancreatic IB PLA2 activity in the presence of the test compound (relative to what is detected in the absence of the test compound) is indicative of the test compound being a pancreatic IB PLA2 modulating agent.

The efficacy of the modulating agent can be assessed by generating dose response curves from data obtained using various concentrations of the test modulating agent. Moreover, a control assay, such as a PLA2 activity method disclosed herein, can also be performed to provide a baseline for comparison.

In another embodiment, the assay is a cell-free assay in which pancreatic IB PLA2 is contacted with a test compound and the ability of the test compound to modulate (e.g., stimulate or inhibit) the activity of the pancreatic IB PLA2 is determined. The pancreatic IB PLA2 protein (and its isologs) can be provided as a gastric secretion, as a purified or semipurified polypeptide, or as a recombinantly expressed polypeptide.

Recombinant expression vectors that can be used for expression of pancreatic IB PLA2 are known in the art (as noted above). In one embodiment, within the expression vector, the pancreatic IB PLA2-coding sequences are operably linked to regulatory sequences that allow for constitutive or inducible expression of pancreatic IB PLA2 in the indicator cell(s). Use of a recombinant expression vector that allows for constitutive or inducible expression of pancreatic IB PLA2 in a cell is preferred for identification of compounds that enhance or inhibit lipid hydrolysis activity of pancreatic IB PLA2. In an alternate embodiment, within the expression vector, the pancreatic IB PLA2 coding sequences are operably linked to regulatory sequences of the endogenous pancreatic IB PLA2 gene (i.e., the promoter regulatory region derived from the endogenous gene). Use of a recombinant expression vector in which pancreatic IB PLA2 expression is controlled by the endogenous regulatory sequences is preferred for identification of compounds that enhance or inhibit the transcriptional expression of pancreatic IB PLA2.

In one embodiment, test compounds are employed in a suitable competitive assay to assess the ability of the test compounds to displace a known bile salt from binding to a pancreatic IB PLA2 polypeptide. In an exemplary competitive assay, a known amount of bile salt is added to a sample containing a pancreatic IB PLA2 polypeptide, and the sample is then contacted with a test compound. Suitable agents effectively compete with the bile salt for binding to the pancreatic IB PLA2 polypeptide. Methods of determining the effectiveness of a test compound are discussed elsewhere herein.

This invention further pertains to novel agents identified by the above-described screening assays. Accordingly, it is within the scope of this invention to further use an agent identified as described herein in an appropriate animal model. For example, an agent identified as described herein can be used in an animal model to determine the efficacy, toxicity, or side effects of treatment with such an agent. Alternatively, an agent identified as described herein can be used in an animal model to determine the mechanism of action of such an agent. Furthermore, novel agents identified by the above-described screening assays can be used for treatments as described herein.

All of the compositions and methods disclosed and claimed herein can be made and executed without undue experimentation in light of the present disclosure. It will be apparent to those of skill in the art that variations may be applied to the compositions and methods and in the steps or in the sequence of steps of the method described herein without departing from the concept, spirit, and scope of the invention. More specifically, it will be apparent that certain agents which are chemically or biologically related may be substituted for the agents described herein while the same or similar results would be achieved. All such similar substitutes and modifications apparent to those skilled in the art are deemed to be within the spirit, scope, and concept of the invention as defined by the appended claims.

### EXAMPLES

The present invention is further defined in the following Examples. It should be understood that these Examples are given by way of illustration only. From the above discussion and these Examples, one skilled in the art can ascertain the preferred features of this invention, and without departing from the spirit and scope thereof, can make various changes and modifications of the invention to adapt it to various uses and conditions.

### Material and Methods

Bile salts and conjugates from Sigma were crystallized in ethanol to >98% purity. Ezetimibe, the active component of Zetia® pills (Schering-Plough), was dissolved in tetrahydrofuran. Preparation and characterization of pig pancreatic IB PLA2 [22] and its (62-66)-deletion [23] and the Trp-substitution mutants [24] has been described. Human pancreatic IB PLA2 was kindly provided by Dr. Suren Tatulian (Orlando, Fla.), and Naja venom PLA2 DEIII was from Bert Verheij (Utrecht, The Netherlands). Several PLA2 rate lowering components of guggul (bark resin of Commiphora mukul) were isolated, and their structures determined by standard methods. Sources of lipids and other reagents as well as rationale for the choice of the experimental conditions and protocols are established before [12, 13, 25, 52]. Specific conditions are given in the text and figure legends.

### Kinetic Measurements

Aqueous dispersion of phospholipid was sonicated in a bath type sonicator. Virtually transparent suspension was annealed for at least 4 hours at room temperature before use [13]. Reaction progress for the PLA2 catalyzed hydrolysis of sonicated DMPC (1 mM) vesicles was measured by pH-stat titration (Radiometer) with 1 mM 2-aminopropanediol in 4 ml reaction buffer containing 5 mM CaCl₂ for DMPC (or 0.5 mM for DMPM), 1 mM NaCl, and 0.1 mM EDTA at pH 8.0 and 24° C. in the nitrogen purged closed atmosphere [18, 26, 27]. Reaction was initiated by adding 0.1 μg enzyme in 1-10 μl solution. The pH stat method was also used for monitoring the rate of hydrolysis of DMPM (0.4 mM) vesicles in the presence of polymyxin [27-29], or of the mixed-micelles of POPC with bile salts in 1:2 ratio [13, 30]. With suitable modifications (as disclosed herein), these methods can be used to screen for potential targets for the modulator site on PLA2.

### Fluorescence Measurements

Fluorescence emission spectrum or time course of the intensity change from tryptophan or the resonance energy transfer probe TMA-DPH were measured in the ratio mode on SLM-Aminco AB2 in a 1 cm cuvette [31, 32] in 1.6 ml stirred buffer containing 1 or 2 μM PLA2 in 10 mM Tris, 5 mM CaCl₂ and 20 mM NaCl at pH 8.0 and 24° C. The slit-widths were 4 nm with excitation at 280.

### Isothermal Calorimetric Measurements

ITC titration of PLA2 with <1 mM (below the CMC) bile salt stock solution was carried out on the Microcal calorimeter (model VP-itc) with cell volume of 1.42 ml with stirring at 300 rpm. Successive injections were made every 5 min. Standard software was used for the peak integration. The heat change with the internal calibration associated with the dilution of the titrant in the absence of the buffer was subtracted from the heat change obtained by the injection of the bile salt in the presence of 8.5 μM PLA2. The net heat change per injection in the presence of PLA2 was integrated to obtain the total heat change as a function of the total added amphiphile concentration.

### Characterization of the Guggul Compounds

Guggul resin is believed to lower fat and cholesterol uptake [56B]. Applicants' assays show that crude extract of resin in organic solvent had significant PLA2 rate lowering activity. About 70% of its PLA2 rate-lowering activity is extracted (Soxhlet) with hexane and remains in the nonvolatile fractions. Further fractionation showed that the activity is mainly due to six out of more than 100 components in the hexane extract. The components of guggul resin were fractionated by taking advantage of solubility, crystallization, and chromatography on silica gel and reverse phase column. Guggulosterones, cembrenes and other known as well as unknown compounds were structurally characterized by spectroscopic methods. Results in Table 1 and FIG. 14 show that among the active components characterized so far cembrenes were significantly more active than guggulosterones, which in turn are significantly more active than some of the common progestane hormones.

### Example 1

### Biphasic Effect of Bile Salt on the PLA2 Catalyzed Hydrolysis of DMPC Vesicles

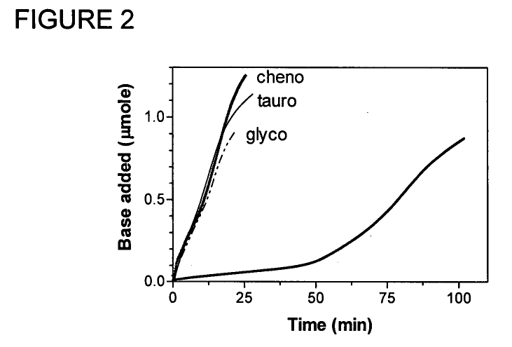

As shown in FIG. 2, the reaction progress for the PLA2 catalyzed hydrolysis of DMPC vesicles is complex and it is significantly altered in the presence of bile salts [13]. As shown in FIG. 2, the major effect of bile salts is to lower the duration of the delay to the stationary phase of the reaction progress and virtually no delay is seen above a critical mole fraction related to the concentration of bile salt and the substrate vesicle. Delay is not seen at higher mole fractions of the bile salts. However, as summarized in FIG. 3, the initial rate depends on the mole fraction and the structure of the bile salt. As also shown in FIG. 2, the delay is <1 min for the rates above 0.025 mM bile salt

Delay before the onset of the stationary phase of the PLA2 catalyzed reaction progress for the hydrolysis of zwitterionic phospholipids has been extensively characterized [11, 12, 14, 16, 33, 34]. Products of hydrolysis, lysophosphatidylcholine and fatty acid, formed during the delay remain in the vesicles, and the delay is not seen with covesicles of the substrate and product. In FIG. 2, the apparent initial rate of PLA2 catalyzed hydrolysis of 1 mM DMPC at 24° C. without added bile salt is 3.5 s⁻¹. With added bile salt, the delay decreases, virtually no delay is seen above 0.025 mM bile salts, and the apparent initial reaction rate is virtually the same as seen after the long delay in the absence of bile salt [13]. The apparent rates in FIG. 3 were obtained from the initial slopes of such reaction progress during which <5% of the total substrate is hydrolyzed. After the rate increase, at 0.02 to 0.04 mM bile salt the peak rate and the rate change at >0.04 mole fraction bile salt is very different for different bile salts. For example, the peak rate above 0.04 mM cholate is 140 s⁻¹ and changes little up to 0.02 mM. On the other hand, with TCDOC the peak rate is 80 s⁻¹ and then decreases at higher concentrations.

The peak rate for the hydrolysis of DMPC vesicles is in the 0.02 to 0.06 mM range for all the bile salts (FIG. 3), and the rate decrease is observed at higher concentrations of certain bile salts. The CMC of the bile salts used in this study range between 0.8 to 5 mM. Neither the peak rate, nor the bile salt concentration for the peak rate, correlates to the CMC. It is also unlikely that these effects are due to disruption of DMPC vesicles to form mixed-micelles. Such disruption of vesicles occurs above 0.3 mole fraction bile salt, whereas at least for cholate as well as TCDOC, urso-DOC and taurourso-DOC the observed changes are essentially complete well below 0.1 mole fraction.

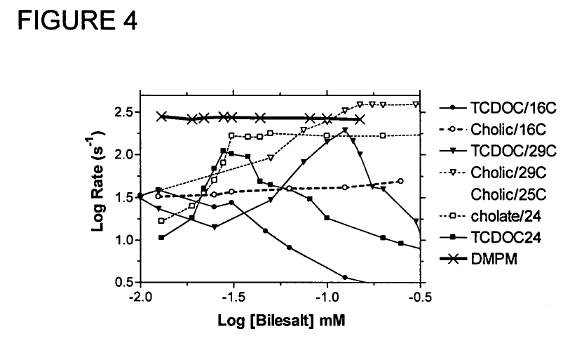

As shown in FIG. 4, bile salts do not have a noticeable effect on the processive interfacial turnover rate in the scooting mode on DMPM vesicles [18, 29] or DC₇PC micelles [35]. These results show that the contribution of the surface dilution by bile salts is small. This is consistent with K_{M}*0.35 mole fraction for DMPM [18] and 0.65 mole fraction for DC₇PC and DMPC [35]. It is also consistent with the kinetic effect of competitive inhibitors. For the hydrolysis of DMPC+cholate, DMPM, or DC₇PC, the mole fraction for 50% inhibition by a competitive inhibitor is not significantly different (results not shown). This is expected if K_{I}* and K_{M}* values on these interface are comparable. Thus, it is unlikely that bile salts have a direct effect on the interfacial turnover parameters K_{M}* or k_{cat}*. In these assays, the products of hydrolysis or the gel fluid phase behavior of DMPM vesicles has little effect on the processive turnover rate [36, 37].

### Example 2

### Additional Boundary Conditions

For the calculation of the mole fraction of the added bile salt, Applicants assumed that bile salts are distributed on both sides of DMPC bilayer. Even if this is not the case, controls show that kinetic effects of bile salt are not due to asymmetric distribution because the observed rates are comparable if bile salt is added before the formation of vesicles. Also, the reaction progress for the hydrolysis of DMPC with or without added bile salt is not noticeably affected by the transmembrane potential induced by gradients of K⁺, Na⁺ or Ca²⁺ ions in the presence of valinomycin, monensin or A23187 (calcimycin), respectively.

The difference between the effect of cholate versus TCDOC is also seen for the PLA2 catalyzed hydrolysis of the mixed micelles of POPC with bile salts. The reaction progress for the hydrolysis of POPC vesicles with <0.5 mole fraction bile salt is complex and could not be interpreted. With suitable controls and calibration curves, mixed micelles of POPC with >0.5 mole fraction bile salt are used for PLA2 assays [30], however the results are not suitable for kinetic analyses of the reaction path [13, 16, 17]. For example, the reaction progress is nearly ideal for the hydrolysis of mixed micelles of 1 mM POPC with 0.7 mole fraction cholate or TCDOC. The maximum rate is in the initial linear region, and the slope decreases monotonically with the substrate depletion and product accumulation. In these mixed-micelles, the initial rate of hydrolysis of POPC is 150 s⁻¹ at 0.7 mole fraction cholate compared to 15 s⁻¹ with 0.7 mole fraction TCDOC. This difference is comparable to the difference seen at 0.1 mole fraction cholate or TCDOC in DMPC vesicles at 24° C. (FIG. 2). These results with POPC also rule out possible anomalous effect of bile salts on the gel-fluid phase change because mixed micelles do not exhibit such a temperature dependent change. Effect of bile salt on the gel-fluid phase of DMPC bilayer change cannot account for the monophasic rate increase with cholate and the biphasic effect of TCDOC. Qualitatively similar effects are observed not only at 24° C. (FIG. 3) but also below and above the gel-fluid coexistence range of 22-25° C. for DMPC vesicles. As shown in FIG. 4, between 16 to 29° C. the maximum rate changes 10-fold. In the presence of TCDOC, the maximum is at 0.013 mM at 16° C. and 0.13 mM at 29° C. Together, results so far show that the differences between the kinetic effects of bile salts on the PLA2 catalyzed hydrolysis of DMPC vesicles do not depend on the bilayer versus micellar organization or the gel-fluid phase behavior of the substrate interface. Applicants attribute the monophasic and biphasic kinetic effects of bile salts on the steps that are not part of the interfacial turnover cycle.

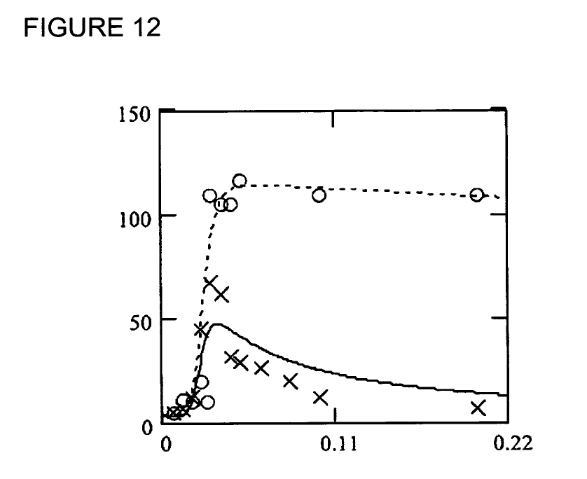

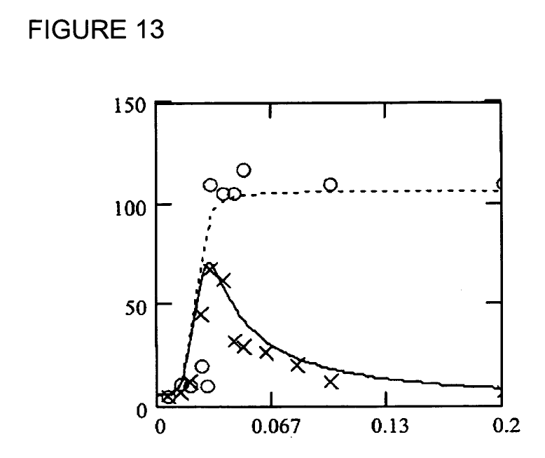

In the model exemplified in FIGS. 12 and 13, the rising phase is attributed to enhanced binding to the interface and k_{cat}*-activation, and the falling phase is to selective binding of certain bile salts to the R-site of PLA2 to form EB complex with different extent of allosteric activation. For detailed studies, Applicants focused on the difference between the effect of cholate versus TCDOC. The rising phase of the fit shown in FIGS. 12 and 13 follow the boundary conditions outlined so far with key assumptions outlined below. The dissociation constant K_{d} for PLA2 bound to DMPC vesicles is 3 mM. K_{d} decreases to 0.2 mM in the presence of 0.1 mole fraction products of hydrolysis [39] or bile, salts [13], and k_{cat}*-activation by the interfacial anionic charge is 15-fold [38]. Such effects for bile salts in DMPC vesicles cannot be modeled because the partition coefficients of bile salts are not known. Also, it is not trivial to model the mole fraction of the accumulated product and its distribution in the vesicle population. Such variables control the interfacial surface charge density that accounts for the loss of the delay before the onset of the stationary phase reaction progress. For the fit in FIGS. 12 and 13, Applicants assumed that, during the stationary phase, vesicles contain 0.08 mole fraction bile salt; in addition, the products formed in situ before the onset of the stationary phase also remains in the substrate interface. These assumptions quantitatively account for the rising phase.

The fit for the falling phase with TCDOC requires additional assumptions. As shown later, binding of TCDOC to the R-site of PLA2 forms an EB complex. Concentration of EB under the kinetic assay conditions would depend on the concentration of B in the aqueous phase. Also, it is necessary to establish whether EB* is formed in the interface and if EB* is catalytically functional. For the fits in FIGS. 12 and 13, Applicants assumed K_{EB} of 5 μM for the complex with TCDOC and >800 μM for the weak complex with cholate. The main effect of the formation of EB is that it lowers the fraction of the enzyme in the interface active E* form. For these fits, it is necessary to make additional assumptions. For the fit in FIG. 12, partitioning of TCDOC as a function of the bile salt is assumed to be non-ideal. Applicants cannot rule out other possibilities including the contribution of other yet unidentified EB* complexes in the interface.

### Example 3

### Effect of TCDOC Depends on the 62-66 Loop of PLA2

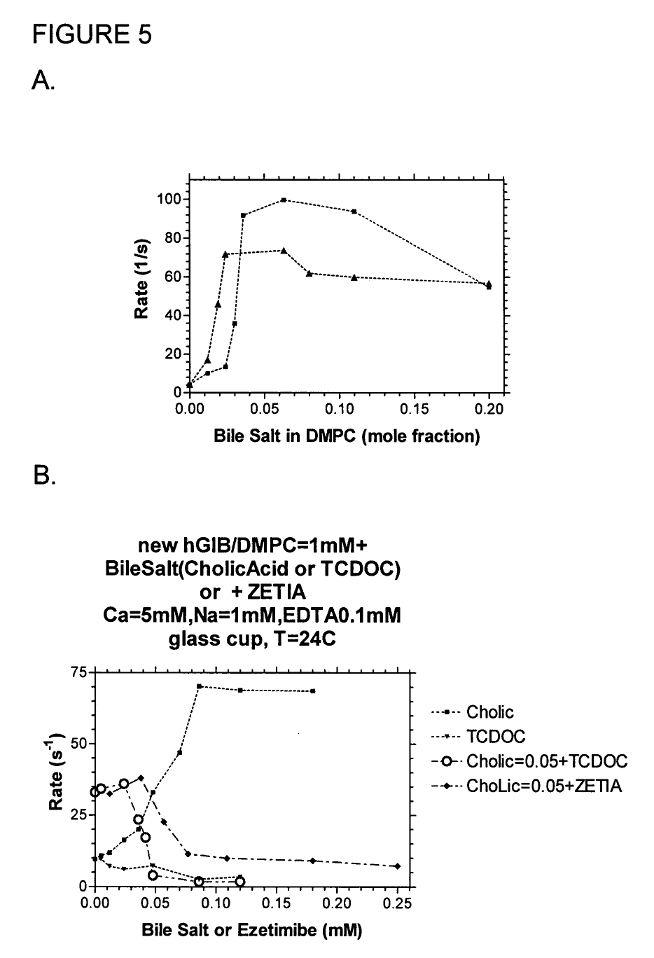

Formation of EB or E*B complex under the kinetic conditions implies that the falling phase would depend not only on the structure of the bile salt (FIG. 3) but also on the structural features of PLA2. Results in FIG. 5A show that the difference between cholate versus TCDOC is not seen with ΔPLA2 in which the 62-66 loop is deleted. Also, DE2 PLA2 from venom of Naja melanoleuca, which is evolutionarily without the 62-66 loop [40], does not distinguish between cholate and TCDOC (results not shown). Also, as shown in FIG. 5B, the rate of hydrolysis of DMPC vesicles by human pancreatic IB PLA2 increases with cholate, and virtually no rate increase is observed with TCDOC. These results suggest that the 62-66 loop in pancreatic IB PLA2 has evolved for the allosteric regulation by the binding of certain bile salts.

### Example 4

### Ezetimibe, TCDOC, and Masticadienolic Acid Lower the Rate of Hydrolysis of DMPC+Cholate

Independent support for selective binding of the rate lowering compounds to a regulatory site on PLA2 comes from the rate-lowering effect of such compounds on the hydrolysis of DMPC vesicles containing 0.05 mole fraction cholate. As shown in FIG. 5B, TCDOC is more effective than ezetimibe in lowering the rate of hydrolysis by human PLA2. On the other hand, as shown in FIG. 6, ezetimibe is more effective in lowering the rate with the pig pancreatic PLA2. Masticadienolic acid is effective by another factor of ten, possibly related to its modest affinity for the active site of PLA2 [64].

Applicants studied the rate lowering effects of many other synthetic compounds and also those isolated from plants alleged to lower fat and cholesterol levels or treat obesity in humans. Results with six active compounds from guggul are summarized in Table 1. Scores of other compounds in guggul were not active. Key results with one of the potent rate-lowering compounds from guggul are shown in FIG. 14 and Table 1. These and other (not shown) results show that the more potent compounds lower the PLA2 catalyzed rate at <0.001 mole fraction in the substrate interface. These compounds are not competitive inhibitors of PLA2; however, some results suggest that it is possible to design compounds that bind to the active site and also to the R-site. Together, significant structural specificity of the enzyme, bile salt and other rate-lowering compounds is consistent with specific interaction of these compounds with PLA2.

TABLE 1
Relative Rate-lowering (%) Activity of compounds^{a}
from Guggul (top six) and others at 0.03 mole fraction in
assay with DMPC + 0.05 mole fraction cholate and human
or pig pancreatic PLA2

| Compound | hPLA2 | pPLA2 |
|---|---|---|
| #6 | 67 | 75 |
| #14 | 96 | 61 |
| #15 | 99 | 72 |
| #16 | 76 | 80 |
| Guggulosterone | 53 | 85 |
| Gugguloster-3-one- | 56 | 90 |
| 16-ol | | |
| TCDOC | 90 | 26 |
| Masticadienolic acid | 66 | >95 |
| Ezetimibe | 5 | 30 |

^{a}These compounds have modest structural differences. Only #16 and guggulosterone are reported to be present in guggul.

### Example 5

### Biphasic Effect of TCDOC on the Binding of PLA2

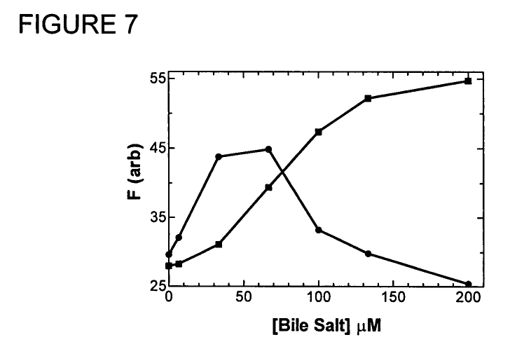

As modeled in FIGS. 12 and 13, changes in K_{d} (for the enzyme bound to the interface) and k_{cat}*-activation of the interfacial chemical step correlate well with the rate increase observed with cholate added to DMPC vesicles. Fluorescence emission from Trp-3 increases on the binding of PLA2 to phospholipid interface [39, 41]. Based on the concentration dependence, K_{d} for PLA2 is about 3 mM for DTPC, and about 20-fold lower in the presence of bile salts and other anionic additives including the products of hydrolysis of phospholipids [12, 13, 31, 39, 42, 43]. As shown in FIG. 7, the change in the Trp-3 emission intensity on the binding of PLA2 to DTPC vesicles containing 5 mole % product is different in the presence of cholate versus TCDOC. The monophasic effect of cholate and the biphasic effect of TCDOC correlate with their effects on the rate of hydrolysis on DMPC (FIG. 3). One of the simplest interpretations is that both the kinetic effects in FIG. 3 and the fluorescence changes in FIG. 7 are due to the change in the fraction of the total enzyme in the interface active E* form. Thus, ignoring the quantitative differences and second order effects, the decrease in the emission intensity at the higher TCDOC concentration is expected if the bound enzyme is desorbed due to the formation of EB complex in the aqueous phase. However, the fluorescence decrease could also be observed if Trp-3 emission intensity of E*B formed at the interface is lower.

### Example 6

### Effect of Cholate and TCDOC on PCU Binding

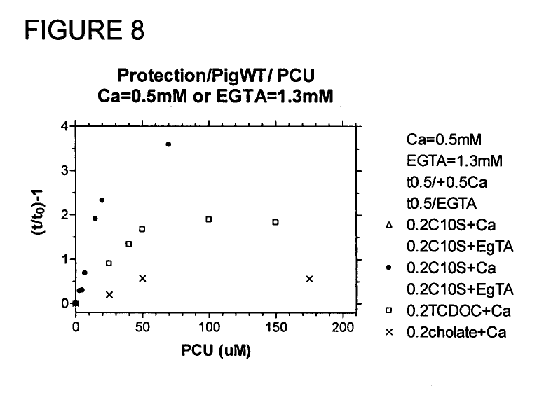

The calcium dependent binding of an active site directed inhibitor PCU to PLA2 increases the halftime for the alkylation of the catalytic residue His-48 [26, 44, 45]. As shown in [45], binding of a molecule of PCU or decylsulfate to the regulatory R-site allosterically increases the affinity of PCU for the active site >30-fold. The alkylation time increase <2-fold in the presence of 0.2 mM TCDOC or cholate with or with calcium suggesting a modest effect on the reactivity of His48. Also, accessibility of Trp-3 for oxidation by N-bromosuccinimide [23] does not change noticeably in the presence of monodisperse bile salts. On the other hand, as shown in FIG. 8, in the presence of cholate and TCDOC, the effect of PCU concentration on the alkylation time is different. In particular, the alkylation time for the complex depends on the amphiphile bound to the regulatory site. The protection offered by PCU bound to the active site is decreased in the order PCU>decylsulfate>TCDOC>cholate bound to the R-site. These results suggest that not only is the binding specificity for the R site poor, but the reactivity of His-48 in the complex also depends on the amphiphile bound to the R-site. Applicants suggest that such differences are due to the allosteric effects that control the chemical step.

### Example 7

### Binding of Bile Salt to the R-Site

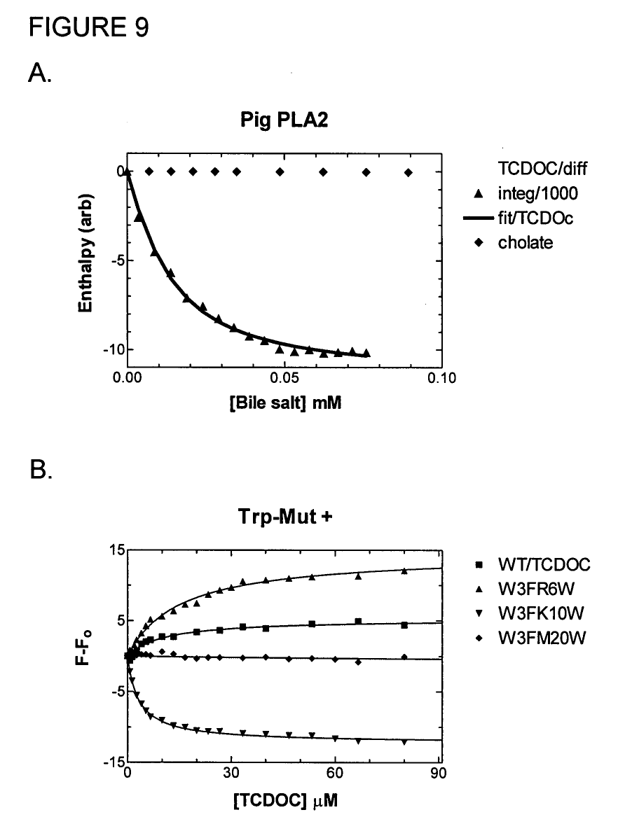

Several independent methods showed that there is significant difference in the interaction of PLA2 with cholate versus TCDOC. As shown in FIG. 9A, on the titration of PLA2, significant enthalpy change is observed only with TCDOC but not with cholate. The change with TCDOC could be fitted to a single hyperbola with K_{EB} of 9 μM and Hill coefficient 1, which is consistent with the formation of 1:1 complex of PLA2. As also summarized in the legend to FIG. 9A, K_{EB} values for the complexes with TUDOC, TDOC or UDOC are in 2 to 38 μM range with the enthalpy change in −12 to −16 kcal/mole range suggesting that the formation of EB is largely enthalpy driven with modest entropic contribution. Note that bile salts with lower K_{EB} values are also more effective in lowering the rate in the falling phase (FIG. 3).

As shown in [45], the binding of decylsulfate to the R site is accompanied by a modest increase in the emission intensity from Trp-3. As shown in FIG. 9B, the TCDOC concentration dependent change in the fluorescence emission intensity from the Trp-substitution mutant of PLA2 depends on the position of the fluorophore. As summarized in Table 2, the value of K_{EB} depends on the position of Trp, although Hill coefficient remains 1. Cholate did not show the intensity change with any of the Trp-mutants; however, K_{EB} is different for the complex with TCDOC and TDOC with the Trp-mutants. These results show that the K_{EB}, as well as the direction and the magnitude of the intensity change, depend on the position of the Trp-substituent and also the position of the structural features of the bile salt (FIG. 1). Trp-substituents near the N-terminus have significant effect on K_{EB}, which suggests that the (1, 10)-helix plays a significant role in the specific binding of the bile salt to form EB complex. Note that K_{EB} for the EB complex of ΔPLA2 is about 7-fold larger than that for PLA2. It is qualitatively consistent with the result in FIG. 5, but quantitative fit suggests that the change in K_{EB} alone cannot account for the difference between the kinetic effects of TCDOC on the hydrolysis of DMPC by PLA2 and Δ62-66.

TABLE 2
Binding parameters for the binding of TCDOC and TDOC to Trp-
mutants of PLA2

| | TCDOC | Tauro-DOC |
|---|---|---|
| Mutant | K_{EB} | K_{EB} |
| (3W)(WT) | 6 | 30 |
| proPLA2 | 27 | — |
| Δ62-66 | 41 | — |
| R6M | 150 | >100 |
| K10M | 8 | >100 |
| R6M/K10M | — | 10 |
| A1W/W3F | 210 | — |
| L2W/W3F | 18 | 0.02 |
| R6W/W3F | 50 | 4 |
| K10W/W3F | 4 | 10 |
| L19W/W3F | 3 | 44 |
| M20W/W3F | 75 | 2 |
| K53W/W3F | 15 | 33 |
| K56W/W3F | 21 | 35 |
| K63W/W3F | 3 | — |
| K87W/W3F | 200 | 2 |
| N117W | 7 | — |

### Example 8

### Bile Salts Quench RET Intensity from TMA-DPH Bound to PLA2

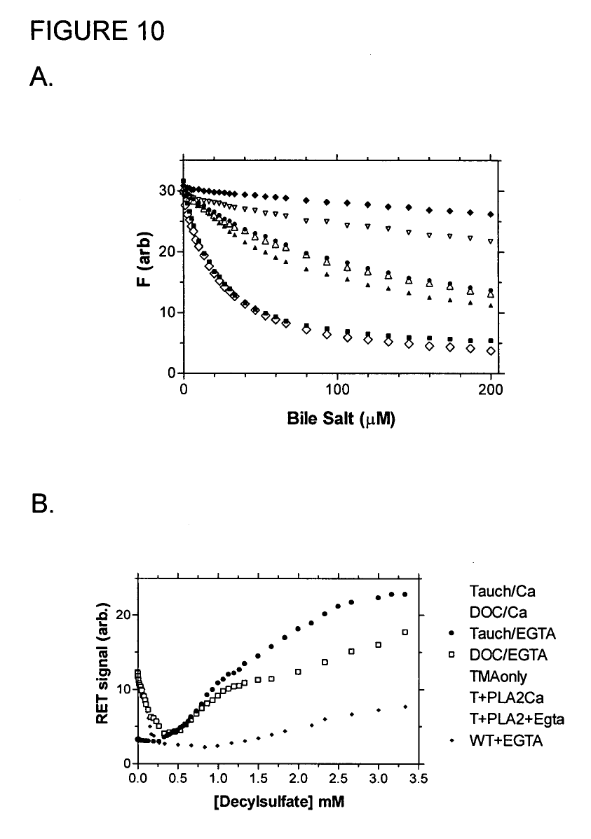

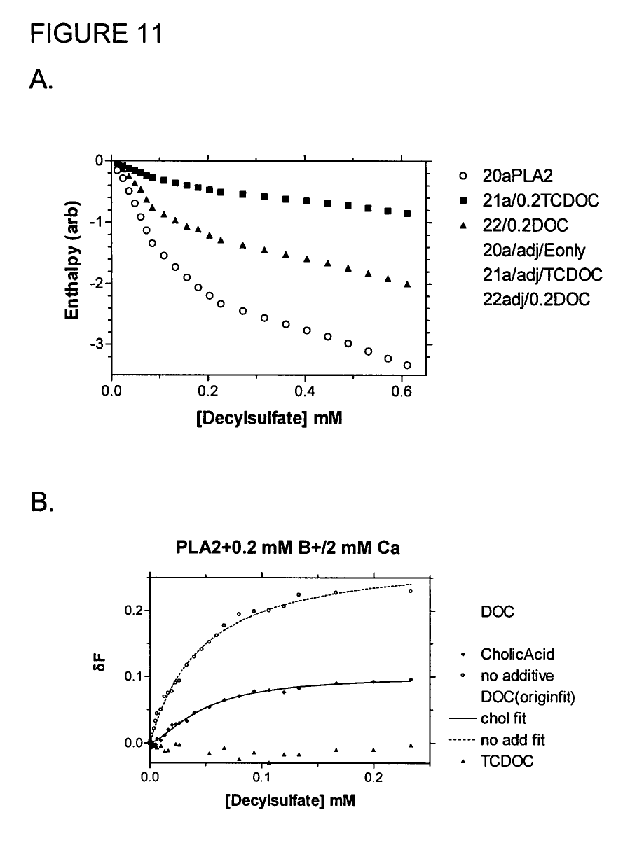

As shown in FIG. 10A, the fluorescence resonance energy transfer signal from TMA-DPH bound to PLA2 decreases with the concentration of bile salts. The shape of the titration curve depends on the structure of bile salt, but the signal at saturating bile salt concentration does not appear to return to the baseline level for TMA-DPH seen in the absence of PLA2. Applicants' interpretation is that TMA-DPH remains bound to the EB complex. As expected for the binding of bile salt to the R-site, calcium is not required for the quenching. As discussed in [45], these results provide only qualitative evidence for the occupancy of the R-site. However, efficacy of bile salts to quench TMA-DPH bound to PLA2 correlates with their ability to lower the PLA2 catalyzed rate of hydrolysis of DMPC vesicles (FIG. 3), i.e. the rank ordering for the bile salts in FIG. 10A is roughly the same and cholate is least effective and TUDOC and TCDOC are most effective. These results do not necessarily show that that TMA-DPH and bile salt bind to the same site on PLA2. However, as shown in FIG. 11A of [45], the RET signal from TMA-DPH bound to Δ62-66 PLA2 is significantly smaller compared to WT, and also the quenching with added bile salt is small. These results are also consistent with a role of the 62-66 loop in the lowering of the rate on the binding of certain bile salts to WT (FIG. 5A).

### Example 9

### Occupancy of the R-Site and the Binding of Decylsulfate to the I-Face

There is a complex relationship between the interactions with the R-site, i-face and the active site of PLA2. As shown in FIG. 10B, the titration curve for PLA2+TMA-DPH with monodisperse decylsulfate is noticeably different in the presence of TDOC versus deoxycholate, and the difference is comparable to that observed for TCDOC versus cholate. The RET signal intensity from TMA-DPH bound to PLA2 is significantly lower in the presence of TDOC as if it quenches the RET signal. On the other hand, the initial signal intensity is not influenced in the presence of 0.2 deoxycholate, and the intensity decreases with <0.3 mM decylsulfate. In conjunction with results in [45], results here show that the binding of rate-lowering bile salts to PLA2 overlap of the binding of PCU and decylsulfate to the R-site [65, 66]. Results in FIG. 11A also show that the bile salts also influence the binding of decylsulfate to the i-face to form E₂# complex.

Results in FIGS. 11A and 11B show that both TCDOC and cholate influence the effect of decylsulfate on PLA2. Isothermal calorimetric titration results in FIG. 11A show that the magnitude of the exothermic enthalpy change associated with the binding of decylsulfate to PLA2 is lower in the presence of 0.2 mM cholate, and more so in the presence of 0.2 mM TCDOC. As shown in FIG. 11B, the change in the Trp-3 emission intensity also shows that although the Trp-environments are different in the presence of the bile salts, at least cholate does not change the affinity of decylsulfate for the R-site. For example, as summarized in Table 3, the main effect of the presence of bile salt is on the Trp-signal intensities rather than the dissociation constant for decylsulfate.

TABLE 3
Effect of 0.2 mM TCDOC on the Decylsulfate Binding Parameters to
Wild-Type PLA2 and its W3F/R6W Mutants

| | | | | | 6W + |
|---|---|---|---|---|---|
| Parameters | 3W | 3W + Chol | 3W + TCDOC | 6W | TCDOC |
| K₁^{#} | 0.05 | 0.06 | — | 0.05 | — |
| K₂^{#} | 0.65 | — | 0.54 | 0.51 | 0.65 |
| a₁ | 0.26 | 0.10 | −0.03 | −0.07 | −0.09 |
| a₂ | 0.56 | — | −0.07 | −0.36 | −1.1 |
| n₁ | 1.7 | 1.5 | — | 2.7 | — |
| n₂ | 8 | — | 6 | 2.5 | 2.0 |

### REFERENCES

[1] Homan R & Krause B R, Curr. Pharm. Des. 3:29-44 (1997).
[2] Mansbach C M et al., Intestinal Lipid Metabolism, Kluwer Academic Press, Plenum Publishers, New York (2000).
[3] Richmond B L et al., Gastroenterology 120:1193-1202 (2001).
[4] Verheij et al., Rev. Physiol. Biochem. Pharmacol. 91:91-203 (1981).
[5] Homan R & Jain M K, Biology, pathology and interfacial enzymology of pancreatic phospholipase A2, Kluwer Academic Press, Plenum Publishers, New York (2000).
[6] Thomson A B R et al., Can. J. Physiol. Pharmacol. 67:179-91 (1989).
[7] Rosenblum S B et al., J. Med. Chem. 41:973-80 (1998).
[8] Earl J & Kirkpatrick P, Nat. Rev. Drug Discov. 2:97-98 (2003).
[9] Homan R & Hamelehle K L, J. Lipid. Res. 39:1197-1209 (1998).
[10] Huggins K W et al., Am. J. Physiol. Endocrinol. Metabl. 283:E994-E1001 (2002).
[11] Upreti G C & Jain M K, J. Membr. Biol. 55:113-21 (1980).
[12] Apitz-Castro R et al., Biochim. Biophys. Acta 688:349-56 (1982).
[13] Jain M K et al., Biochemistry 32:8360-67 (1993).
[14] Jain M K & De Haas G H, Biochim. Biophys. Acta 736:157-62 (1983).
[15] Jain M K et al., Biochim. Biophys. Acta 980:23-32 (1989).
[16] Berg O G & Jain M K, Interfacial Enzyme Kinetics, Wiley, London, 2002.
[17] Berg O G et al., Chem. Rev. 101:2613-54 (2001).
[18] Berg O G et al., Biochemistry 30:7283-97 (1991).
[19] Clader J W et al., J. Med. Chem. 39:3684-93 (1996).
[20] Kramer W et al., J. Biol. Chem. 280: 1306-20 (2005).
[21] Davis H R et al., J. Biol. Chem. 279:33586-92 (2004).
[22] Seedorf U et al., Biochem. Biophys. Res. Commun. 320:1337-41 (2004).
[23] Tsai Y et al., Biochim. Biophys. Acta 1758:653-65 (2006)
[24] Thunnissen M M et al., Nature 347:689-91 (1990).
[25] Yu B Z et al., Biochemistry 38:4875-84 (1999).
[26] Jain M K et al., Biochemistry 30:10256-68 (1991).
[27] Jain M K et al., Biochemistry 30:7340-48 (1991).
[28] Cajal Y et al., Biochem. Biophys. Res. Commun. 210:746-52 (1995).
[29] Cajal Y et al., Biochemistry 35:299-308 (1996).
[30] Nieuwenhuizn W et al., Eur. J. Biochem. 40:1-7 (1973).
[31] Jain M K et al., Biochim. Biophys. Acta 860:448-61 (1986).
[32] Jain M K & Maliwal B P, Biochemistry 32:11838-46 (1993) (published erratum appears in Biochemistry 33:8618 (1993)).
[33] Yu B Z et al., Biochim. Biophys. Acta 1712:137-51 (2005)
[34] Jain M K et al., Biochim. Biophys. Acta 818:356-64 (1985).
[35] Berg O G et al., Biochemistry 36:14512-30 (1997).
[36] Jain M K et al., Biochim. Biophys. Acta 860:435-47 (1986).
[37] Jain M K et al., Biochim. Biophys. Acta 860:462-74 (1986).
[38] Yu B Z et al., Biochemistry 39:12312-23 (2000).
[39] Jain M K et al., Biochim. Biophys. Acta 688:341-48 (1982).
[40] van Eijk J H et al., Eur. J. Biochem. 132:183-88 (1983).
[41] Jain M K & Zakim D, Biochim. Biophys. Acta 906:33-68 (1987).
[42] Jain M K & Vaz W L, Biochim. Biophys. Acta 905:1-8 (1987).
[43] Jain M K & Jahagirdar D V, Biochim. Biophys. Acta 814:313-18 (1985).
[44] Yu B Z et al., Biochemistry 32:6485-92 (1993).
[45] Jain M K et al., Biochemistry 30:7306-17 (1991).
[46] Friden P M et al., Science 259:373-77 (1993).
[47] Davis et al., Enzyme Eng. 4:169-73 (1978).
[48] Burnham N L, Am. J. Hosp. Pharm. 51:210-18 (1994).
[49] Amselem S et al., Chem. Phys. Lipids 64:219-37 (1993).
[50] Lee B I et al., Biochemistry 38:7811-18 (1999).
[51] Beiboer S H et al., Eur. J. Biochem. 231:747-53 (1995).
[52] Tsai Y et al., Biochim. Biophys. Acta 1758:653-65 (2006).
[52A] Yu B-Z et al., Biochim. Biophys. Acta, In press (2007)
[53] Fernandez M L & Volek J S, Nutr. Metab. 3:17-22 (2006).
[54] Stolk M F et al., Clin. Cardiol. 29:52-55 (2006).
[55] Yatskar L et al., Am. J. Med. 119:285-86 (2006).
[56] Orso E et al., Cytometry A 69:206-08 (2006).
[56A] Schmitz G et al., Curr. Opin. Lipidol. 18:164-72 (2007).
[56B] Urizar N L & Moore D D, Ann. Rev. Nutr. 23:303-13 (2003).
[56C] Bays H & Stein E A, Curr. Opin. Pharmacother. 4:1901-38 (2003).
[56D] Vicens M et al., Bioorg. Med. Chem. 15:2359-67 (2007).
[56E] Kuipers F et al., Curr Opin. Lipidol. 18, 289-297 (2007).
[56F] Tius M A, Chem. Rev. 88, 719-32 (1988).
[56G] Jain M K, & Jahagirdar D V, Biochem. J. 227, 789-794 (1985).
[57] Kunkel T A, Proc. Natl. Acad. Sci. USA 82:488-92 (1985)
[58] Kunkel T A et al., Meth. Enzymol. 154:367-82 (1987)
[59] Walker J M & Gaastra W, eds., Techniques in Molecular Biology, MacMillan Publishing Company, New York (1983)
[60] Hope W C et al., Inflammation 14:543-59 (1990)
[61] Jain M K & Gelb M H, Meth. Enzymol. 197:112-25 (1991)
[62] Oinuma H et al., J. Med. Chem. 34:2260-67 (1991)
[63] Yuan W et al., Biochemistry 29:6082-94 (1990)
[64] Jain M K et al., Phytochemistry 39:537-47 (1995)
[65] Berg O G et al., Biochemistry 43:7999-8013 (2004)
[66] Yu B Z et al., Biochemistry 42:6293-6301 (2003)
[67] Goldberg A C et al., Am. J. Cardiol. 97:376-79 (2006)
[68] McPherson T B et al., J. Pharm. Pharmacol. 57:889-96 (2005)

We claim:

1. A method for up or down regulation of fat and/or cholesterol uptake from the intestine comprising administering to a mammal in need thereof an effective amount of a regulator of pancreatic IB PLA2 functionality.

2. The method of claim 1, wherein said regulator of pancreatic IB PLA2 functionality is an allosteric effector of pancreatic IB PLA2.

3. The method of claim 1, wherein said regulator of pancreatic IB PLA2 functionality effects its regulation through the 62-66 loop region of pancreatic IB PLA2.

4. The method of claim 3, wherein said regulator of pancreatic IB PLA2 functionality is a bile salt or a molecular mimic thereof with steroid, terpene, cembrene, or calixerene skeleton.

5. The method of claim 4, wherein said regulator of pancreatic IB PLA2 functionality is taurochenodeoxycholate, taurodeoxycholate, ursodeoxycholate, masticadienoic acid, masticadienolic acid, guggulsterone, cembrene or a derivative thereof.

6. The method of claim 4, wherein the regulator of pancreatic IB PLA2 functionality is a compound of the formula: 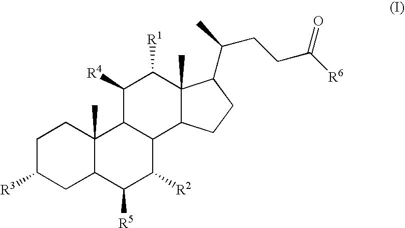
wherein R¹ is H, OH, O, or Ac;
R² is H, OH, O, or Ac;
R³ is OH or O;
R⁴ is H, OH, or O;
R⁵ is H, OH, O, or Ac;
and R⁶ is COOH, CONH₂, SO₄, PO₄, CO-taurine, CO-glycine, CO—NH—(CH₂)ₙ-anion glucuronate, wherein the anion is COOH, —O-phosphate, C-phosphate, O-sulfate, or —C-sulfate and n is an integer in the range of 8 to 12.

7. The method of claim 1, wherein said method is employed to reduce fat and/or cholesterol uptake from the intestine.

8. The method of claim 7, wherein said method is employed in animals consuming high-fat diets.

9. The method of claim 1, wherein said method is employed to increase fat and/or cholesterol uptake from the intestine.

10. A method for regulation of plasma fat and/or cholesterol levels comprising administering to a mammal in need thereof an effective amount of a regulator of pancreatic IB PLA2 functionality.

11. The method of claim 10, wherein said method is employed to reduce plasma fat and/or cholesterol levels.

12. The method of claim 10, wherein said method is employed to increase plasma fat and/or cholesterol levels.

13. A method of regulating the function of a polypeptide of interest comprising:
(a) inserting a 62-66 loop region of a pancreatic IB PLA2 amino acid sequence into the polypeptide of interest; and
(b) administering an effective amount of a regulatory molecule that effects its regulation through said 62-66 loop region.

14. The method of claim 13, wherein said polypeptide of interest is a non-pancreatic IB PLA2 peptide.

15. The method of claim 13, wherein said method is employed to reduce protein functionality.

16. The method of claim 13, wherein said method is employed to increase protein functionality.

17. A pharmaceutical composition for the regulation of uptake of fat and/or cholesterol from the intestine or for the regulation of plasma fat and/or cholesterol levels comprising an effective amount of an isolated bile salt compound.

18. The pharmaceutical composition of claim 17, wherein said isolated bile salt compound is taurochenodeoxycholate, taurodeoxycholate, glycocholate, glycodeoxycholate, tauroursodeoxycholate, masticadienoic acid, masticadienolic acid, guggulsterone, cembrenes, or a mimic thereof.

19. The pharmaceutical composition of claim 18, wherein said cembrene has the formula: 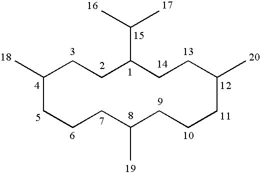
and further wherein said cembrene has 3 to 5 double bonds and is optionally substituted with at least one hydroxyl group, carbonyl group, acetyl group, and/or calixerene ring structure with fused aromatic rings at any of the twenty carbon positions.

20. The pharmaceutical composition of claim 19, wherein said cembrene has modifications selected from the group consisting of double bonds at positions 3-4, 7-8, 11-12, and 15-16; double bonds at positions 2-3, 4-5, 7-8, and 11-12; double bonds at positions 3-4, 7-8, and 11-12; and double bonds at positions 34, 7-8, and 11-12.

21. The pharmaceutical composition of claim 17, further comprising ezetimibe, cholate, a statin, nicotinic acid, a fibrate, a bile acid-binding resin, agonist or antagonist of FxR, or a pancreatic IB PLA2 competitive inhibitor.

22. The pharmaceutical composition of claim 17, wherein said isolated bile salt compound has the formula: 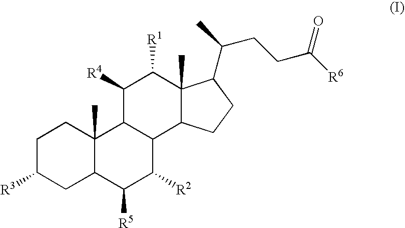
wherein R¹ is H, OH, O, or Ac;
R² is H, OH, O, or Ac;
R³ is OH or O;
R⁴ is H, OH, or O;
R⁵ is H, OH, O, or Ac;
and R⁶ is COOH, CONH₂, SO₄, PO₄, CO-taurine, CO-glycine, CO—NH—(CH₂)ₙ-anion glucuronate, wherein the anion is COOH, —O-phosphate, C-phosphate, O-sulfate, or —C-sulfate and n is an integer in the range of 8 to 12.

23. A method for determining activity of pancreatic IB PLA2 enzyme in a sample comprising:
(a) combining said sample with a regulator of pancreatic IB PLA2 functionality; and
(b) determining the amount of pancreatic IB PLA2 present as a function of the ability of the enzyme to digest lipids in the presence of said regulator.

24. The method of claim 23, wherein said sample is a biological sample.

25. The method of claim 24, wherein said biological sample is an animal extract.

26. The method of claim 24, wherein said biological sample is a cell culture extract.

27. A method for detecting altered pancreatic IB PLA2 function in a sample comprising:
(a) combining said sample with a regulator of pancreatic IB PLA2 functionality; and
(b) determining the effectiveness of pancreatic IB PLA2 present in said sample as a function of the ability of the enzyme to digest lipids in the presence of said regulator.

28. A method for identifying an agent suitable for regulating pancreatic IB PLA2 enzyme functionality comprising:
(a) combining said agent with pancreatic IB PLA2; and
(b) determining the ability of the agent to regulate pancreatic IB PLA2 functionality as a function of the ability of the enzyme to digest lipids in the presence of said agent.

29. The method of claim 28, wherein the method further employs at least one additional regulatory or non-regulatory compound.

30. The method of claim 29, wherein said additional regulatory compound is a regulator of pancreatic IB PLA2 functionality.

31. A compound of the formula: 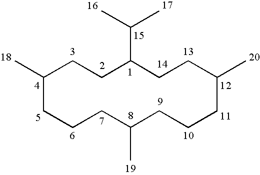
having modifications selected from the group consisting of double bonds at positions 3-4, 7-8, 11-12, and 15-16; double bonds at positions 2-3, 4-5, 7-8, and 11-12; double bonds at positions 3-4, 7-8, and 11-12; and double bonds at positions 3-4, 7-8, and 11-12; said cembrene further optionally comprising a hydroxyl group, carbonyl group, acetyl group, and/or calixerene ring structure with fused aromatic rings at any of the twenty carbon positions.
